(19) United States
(12) Patent Application Publication
(10) Pub. No.: US 2018/0014828 A1
(43) Pub. Date: Jan. 18, 2018

(54) SHAPE MEMORY ALLOY SUTURES AND PROSTHESIS

(71) Applicant: Arthrex, Inc., Naples, FL (US)

(72) Inventors: Matthew Fonte, Concord, MA (US); Matthew Palmer, Medford, CA (US); Robert Devaney, Auburndale, MA (US); Paul Fein, Lexington, MA (US)

(21) Appl. No.: 15/646,385

(22) Filed: Jul. 11, 2017

Related U.S. Application Data
(60) Provisional application No. 62/361,304, filed on Jul. 12, 2016.

(51) Int. Cl. A61B 17/06; A61B 17/00

(52) U.S. Cl. CPC A61B 17/06166; A61B 2017/00871; A61B 2017/0618

(57) ABSTRACT

A suture filament is suitable for use as a suture or a ligature. The suture filament includes a cover formed of a plurality of braided fibers of polyester, and/or ultrahigh molecular weight polyethylene, and/or a plurality of shape memory alloy filaments. The suture filament includes a core of shape memory alloy that is surrounded by the cover.

### CROSS REFERENCE TO RELATED APPLICATIONS

This patent application claims priority to and the benefit of pending prior U.S. Provisional Patent Application Ser. No. 62/361,304, filed on Jul. 12, 2016, the entire disclosure of which is hereby incorporated by reference.

### FIELD OF THE INVENTION

The present invention relates to the use of shape memory alloys to repair tendon and ligament injuries or deformities that require the detachment and reattachment of tendon or ligament to bone or to itself. Specifically, the present invention relates to the use of high strength surgical suture materials. More specifically, the present invention relates to a suture having a shape memory alloy core and a polyester and/or ultrahigh molecular weight polyethylene braided cover that has high strength, elasticity, and elastic properties equal to that of the target soft-tissue, including tendon and ligament. The present invention also relates to shape memory alloy tendon or ligament replacement constructs (prosthesis) that use shape memory alloys and their inherent superelasticity to match the material properties of the target tissue. Surgical techniques include the reattachment of soft-tissue to soft-tissue and the anchoring of soft-tissue to bone.

### BACKGROUND OF THE INVENTION

Suture strength is an important consideration in any surgical suture material. Some of the strongest materials used as a suture include metal strands, such as stainless steel. However, these materials can be difficult to tie and are susceptible to kinking during handling. An ideal suture material is as strong as possible, but also easy to manipulate. Nitinol used as a suture material would need the temperature to be maintained below body temperature during handling and implantation and would need the temperature to be elevated post implantation with a separate external device. This is a cumbersome addition to the surgical process.

Shape memory alloys (SMA), such as Nickel-titanium or Nitinol (NiTi), are functional materials whose shape and stiffness can be controlled with temperature and cold work or heat treatment steps during manufacturing the final product. The metal undergoes a complex crystalline-to-solid phase change called martensite-austenite transformation. As the metal in the high-temperature (austenite) phase is cooled, the crystalline structure enters the low-temperature (martensite) phase where it can be easily bent and shaped. As the metal is reheated above its transition temperature, its original shape and stiffness are restored. Shape memory alloy materials exhibit various characteristics depending on the composition of the alloy and its thermal-mechanical work history. Shape memory alloys can recover large strains in two ways: shape memory effect (SME) and pseudoelasticity, also known as superelasticity (SE). The NiTi family of alloys can withstand large stresses and can recover strains near 8% for low cycle use or up to about 2.5% strains for high cycle use.

As stated above, shape memory alloys show two unique capabilities: shape memory effect and superelasticity, which are absent in traditional materials. Both shape memory effect and superelasticity largely depend on the solid-solid, diffusionless phase transformation process known as martensitic transformation (MT) from a crystallographically more ordered parent phase (austenite) to a crystallographically less ordered product phase (martensite). The phase transformation (from austenite to martensite or vice versa) is typically marked by four transition temperatures, named as Martensite finish (Mf), Martensite start (Ms), Austenite finish (Af), and Austenite start (As) (temperatures are Mf<Ms<As<Af). Therefore, a change in the temperature in the range of Ms<T<As induces no phase change, and both martensite and austenite may coexist if the temperature is in the range of Mf<T<Af. As shown in FIG. 1, the phase transformations may take place depending on changing the temperature (the shape memory effect) or changing the stress (the superelasticity).

### Controlling the Transition Temperatures and Stress Plateaus of Shape Memory Alloy

The transition temperature of shape memory alloys can be controlled through heat treatment. In many cases, the Af temperature should be close to the body temperature (37° C.). For Nitinol, a starting material is available where the Af temperature is around body temperature. However, the transformation temperatures may change because of any cold work and heat treatment steps used when manufacturing the final product.

It is possible to return the Nitinol to its fully annealed state by heating it to 800° C. to 850° C. for 15 to 60 minutes to erase all thermomechanical processing. After this, the Af temperature can be reset by aging the material. The Af temperature is effected by the exact matrix composition. As shown in the Nitinol phase diagram of FIG. 2, as the aging temperature and time increases, nickel rich precipitation reactions occur. This changes how much nickel is in the NiTi lattice. By reducing the amount of nickel in the matrix, aging increases the transformation temperature.

A TTT (time-temperature transformation) diagram can be used to determine the temperature and the period of time at which to age the Nitinol material to achieve an appropriate Af. As seen in the time-temperature transformation diagram of FIG. 3, aging the Nitinol material at 400° C. for approximately 30 minutes results in an Af close to 37° C. The exact Af temperature can be measured by using a differential scanning calorimeter.

As shown in FIG. 4, the temperature differential between (i) the body that the Nitinol device will be installed into (assumed to be 37° C., the temperature of a human body) and (ii) the Af temperature of the shape memory material of the Nitinol device affects the unloading characteristics of the Nitinol device. If there is a smaller temperature differential between the two temperatures, the device will generate a smaller compressive load. Conversely, if there is a larger temperature differential between the two temperatures, the device will generate a larger compressive load.

As shown in FIG. 5, in addition to the Af temperature of the Nitinol device, the stress levels of the upper plateau and the lower plateau of Nitinol's mechanical hysteresis curve are affected by the amount of “cold work” in the device. Cold work is measured as the reduction in a cross-sectional area that the material experiences as part of the raw material's manufacturing. Rolling or drawing operations are typically used to produce a Nitinol bar, tube and sheet material. Final cold work is typically about 30%-50%. During processing, it is possible to control the amount of cold work in the material through high temperature (e.g., greater than about 600° C.) annealing. High temperature annealing erases some or all thermomechanical processing, stress relieves or recrystallizes the material, and returns the alloy to its ingot properties.

Reduced amounts of cold work result in reduced strength. As the percent of cold work is increased, the stress level of the upper plateau rises, and the stress level of the lower plateau falls. The percentage of cold work in the Nitinol device affects its unloading characteristics. As the percentage of cold work increases, the unloading stress decreases.

### Shape Memory Effect

When the temperature is greater than Af (T>Af), the shape memory alloy is in the parent austenite phase with a particular size and shape. Under stress free conditions, if the shape memory alloy is cooled to any temperature less than Mf (T<Mf), martensitic transformation occurs as the material converts to the product martensite phase. Martensitic transformation is basically a macroscopic deformation process, although no transformation strain is generated due to the so-called self-accommodating twinned martensite.

If a mechanical load is applied to this material, and the stress reaches a certain critical value, the pairs of martensite twins begin “detwinning” (conversion) to the stress-preferred twins. The “detwinning” process is marked by the increasing value of strain with insignificant increases in stress. The multiple martensite variants begin to convert to a single variant, the preferred variant determined by alignment of habit planes with an axis of loading. As the single variant of martensite is thermodynamically stable when the temperature is less than As (T<As), there is no reconversion to multiple variants, and only a small elastic strain is recovered upon unloading, leaving the materials with a large residual strain (apparently plastic).

Next, if the deformed shape memory alloy is heated above Af (T>Af), the shape memory alloy transforms to the parent phase (which has no variants). The residual strain is fully recovered, and the original geometric-configuration is recovered. The material fully recovers and recalls from “memory” its original shape before the deformation. This is known as shape memory effect. However, if some end constraints are used to prevent the free recovery to the original shape, the material generates a large tensile recovery stress, which can be exploited as an actuating force for an active or passive control purpose. Shape memory alloy coatings can be processed via shape memory effect.

### Superelasticity

The second feature of a shape memory alloy is superelasticity (or pseudoelasticity). The superelastic (SE) shape memory alloy has the ability to fully regain the original shape from a deformed state when a mechanical load that causes the deformation is removed. For some superelastic shape memory alloy materials, the recoverable strains can be on the order of 10%. Superelasticity depends on the stress-induced martensitic transformation (SIMT), which in turn depends on the temperature and the stress of the shape memory alloy.

In one example, a shape memory alloy that has been entirely in the parent phase (T>Af) is mechanically loaded. Thermodynamic considerations indicate that there is a critical stress at which the crystal phase transformation from austenite to martensite can be induced. Consequently, the martensite is formed because the applied stress substitutes for the thermodynamic driving force usually obtained by cooling (in the case of shape memory effect). The mechanical load imparts an overall deformation to the shape memory alloy as soon as a critical stress is exceeded.

During unloading, the reverse phase transformation starts from the stress-induced martensitic to the parent phase because of the instability of the martensite at this temperature in the absence of stress (again at a critical stress). When the phase transformation is complete, the shape memory alloy returns to the parent austenite phase. Therefore, superelastic shape memory alloy shows a typical hysteresis loop (known as pseudoelasticity or superelasticity), and if the strain during loading is fully recoverable, it is a closed loop. Stress-induced martensitic transformation (or the reverse stress-induced martensitic transformation) are marked by a reduction of the material stiffness. Usually, the austenite phase has much higher Young's modulus as compared to the martensite phase.

### SUMMARY

In one example, a suture filament is suitable for use as a suture or a ligature. The suture filament includes a cover formed of a plurality of braided fibers of polyester and/or ultrahigh molecular weight polyethylene. The cover includes a plurality of shape memory alloy filaments. A core of Nitinol is also included. The core is surrounded by the cover. The core is selected from the group consisting of a single filament of Nitinol, a multi-filament of Nitinol that is wound, braided or twisted, and a ribbon shaped filament of Nitinol. The core has a material condition of a martensitic shape memory alloy where an austenitic finish temperature is at or above body temperature such that the core remains in a martensitic phase after implantation or an austenitic shape memory alloy where the austenitic finish temperature is below body temperature such that the core maintains superelastic properties after implantation.

A suture filament is suitable for use as a suture or a ligature. The suture filament includes a cover formed of a plurality of braided fibers of polyester, and/or ultrahigh molecular weight polyethylene, and/or a plurality of shape memory alloy filaments. The suture filament includes a core of shape memory alloy that is surrounded by the cover.

In another embodiment according to any of the previous embodiments, the shape memory alloy is Nitinol.

In another embodiment according to any of the previous embodiments, the core is a single filament of the shape memory alloy.

In another embodiment according to any of the previous embodiments, the core is a multi-filament of the shape memory alloy. The multi-filament is wound, braided or twisted.

In another embodiment according to any of the previous embodiments, the core is a ribbon shaped filament of the shape memory alloy.

In another embodiment according to any of the previous embodiments, includes a plurality of suture filaments braided into a bundled, multi-filament super structure.

In another embodiment according to any of the previous embodiments, the core has a material condition of a martensitic shape memory alloy where an austenitic finish temperature is at or above body temperature such that the core remains in a martensitic phase after implantation or an austenitic shape memory alloy where the austenitic finish temperature is below body temperature such that the core maintains superelastic properties after implantation.

In another embodiment according to any of the previous embodiments, the suture filament includes a coating of polytetrafluoroethylene.

In another embodiment according to any of the previous embodiments, the cover includes the plurality of shape memory alloy filaments coated in polytetrafluoroethylene.

In another embodiment according to any of the previous embodiments, the plurality of braided fibers of the cover are polyester or ultrahigh molecular weight polyethylene.

In another example, a construct for tendon/ligament repair includes a body comprised of Z struts, W struts, and center line zigzags and comprises a shape memory alloy.

In another embodiment according to any of the previous embodiments, the shape memory alloy is Nitinol.

In another embodiment according to any of the previous embodiments, the shape memory alloy is coated in polytetrafluoroethylene.

In another embodiment according to any of the previous embodiments, the construct includes internal barbs for grip.

In another embodiment according to any of the previous embodiments, the construct is woven or braided.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 illustrates a schematic of a martensite-austenite phase transformation;

FIG. 2 illustrates a Nitinol phase diagram;

FIG. 3 illustrates a time-temperature transformation diagram of Nitinol;

FIG. 4 illustrates a hysteresis curve of Nitinol;

FIG. 5 illustrates a mechanical hysteresis curve of Nitinol;

FIG. 6a illustrates a suture including a single filament core with a braided cover;

FIG. 6b illustrates a suture including a multiple filament core with a braided cover;

FIG. 6c illustrates a multi strand suture wherein each suture has a single filament core with a braided cover;

FIG. 7 illustrates a suture having a plurality of strands and a larger diameter single strand suture of similar strength contacting a tendon;

FIG. 8 illustrates a cross-sectional view of a suture with Nitinol integrated in a cover;

FIG. 9 illustrates a hysteresis curve for tendons and ligaments during the loading and unloading phases;

FIG. 10 illustrates a hysteresis curve for several materials;

FIG. 11 illustrates a hysteresis curve of a specimen tested in compression and tension;

FIG. 12a illustrates a torn tendon;

FIG. 12b illustrates a repaired tendon;

FIG. 13 illustrates a mesh used to repair a torn rotator cuff in a shoulder;

FIG. 14 illustrates a spacer fabric;

FIG. 15 illustrates different stitches and weaves of the spacer fabric;

FIG. 16a illustrates a tendon/ligament repair device that is delivered unstretched;

FIG. 16b illustrates a tendon/ligament repair device that is delivered stretched;

FIG. 17 illustrates a soft-tissue repair device including Z shaped struts;

FIG. 18 illustrates a soft-tissue repair device including W and Z shaped struts; and

FIG. 19 illustrates a fixation device having integral barbs for gripping tissue.

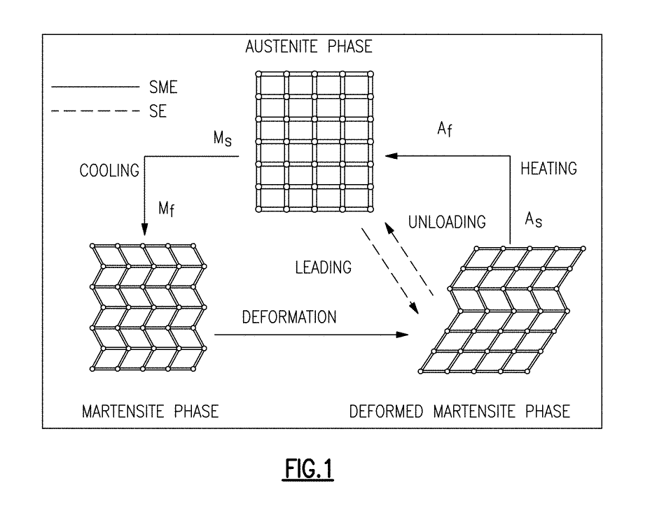

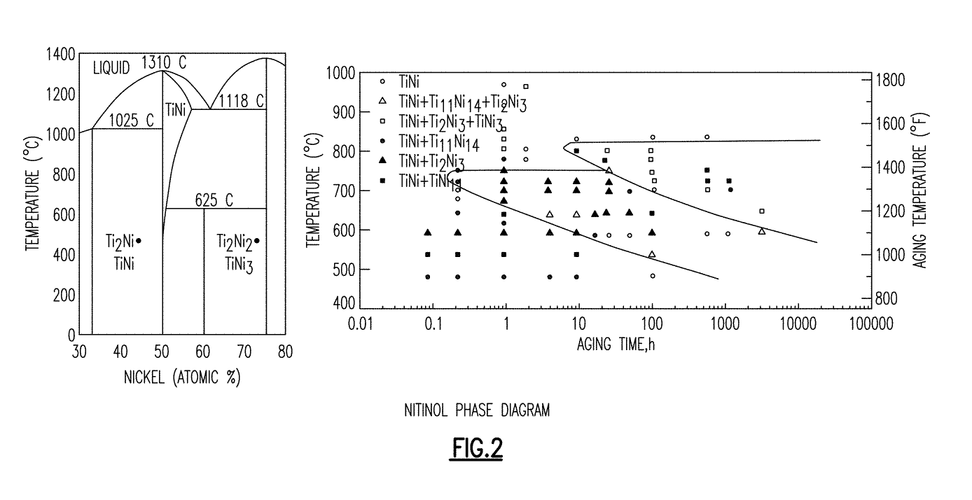

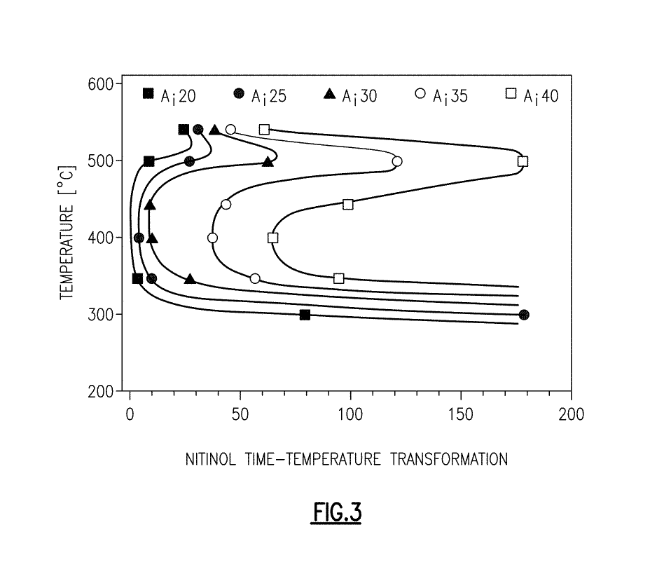

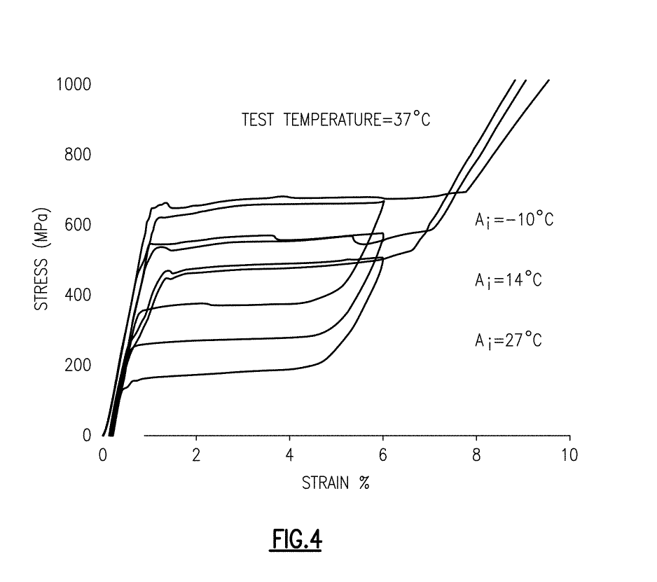

### DETAILED DESCRIPTION

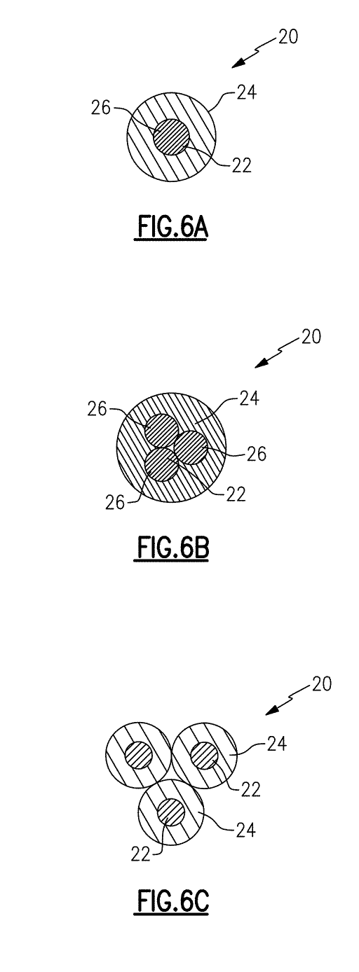

The present invention provides a high strength surgical suture material that has improved strength over conventional suture materials as well as improved manipulation characteristics over conventional metal sutures. In one example shown in FIG. 6a, the suture 20 includes a Nitinol core 22 with a braided cover 24 made of polyester and/or ultrahigh molecular weight polyethylene. Polyethylene provides strength and protects the Nitinol core 22 from abrasion, and polyester provides improved tie down properties.

The preferred suture 20 includes a multi-filament cover 24 formed of a plurality of fibers of polyester and/or ultrahigh molecular weight polyethylene. The cover 24 surrounds a core 22 of a shape memory alloy, such as, but not limited to, nickel-titanium or Nitinol.

The suture 20 of the present invention is ideal for most orthopedic procedures, such as rotator cuff repair, Achilles tendon repair, patellar tendon repair, ACL/PCL reconstruction, hip and shoulder reconstruction procedures, and replacement for suture in knotless anchoring techniques.

Shape memory alloys such as Nitinol are a good supporting suture material where biomechanics or a non-compliant patient require a suture to stretch, particularly in the early healing phase. Nitinol can be stretched up to 8% strain along the material's upper plateau, which can be tuned during manufacturing to match desired soft-tissue characteristics. After unloading up to 2% strain, the force of recovery decreases by more than 50% to the material's lower plateau, such that during the remainder of an unloading phase, the balance of the 6% strain is recovered on the lower plateau. The upper plateau can be tuned to be sufficient to resist stretching of the soft-tissue to soft-tissue interface or the soft-tissue to bone interface for a given suturing method. The reduced lower plateau will prevent damage to the interface during unloading.

The final construct can be engineered to have material properties that match the target tissue. For example, the Achilles tendon has an elastic modulus of approximately 820 MPa, a failure strain of approximately 15% in the tendon substance and 8.5% at the bone-tendon complex, a failure stress of approximately 80 MPa, and a hysteresis value of about 20%. For constructs targeting Achilles tendon repair, material properties fall within this range.

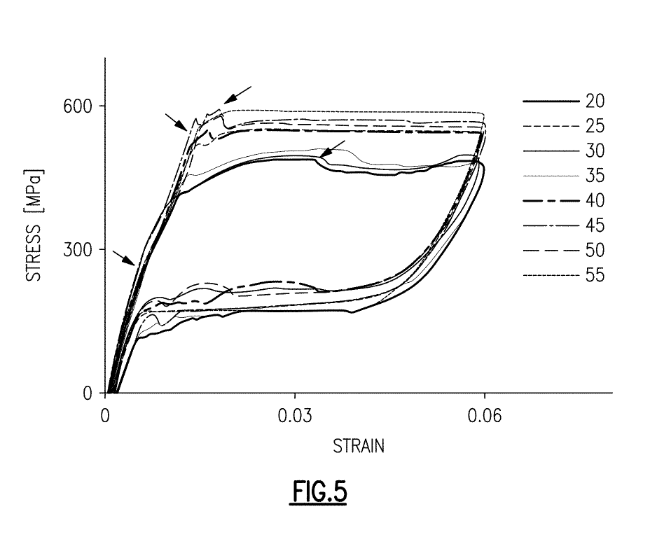

Any construct replacing soft-tissue has to match both the mechanical properties of failure as well as the functional mechanical properties of the soft-tissue. The tendon acts as a viscoelastic element in series with a muscle. Tendon stiffness is critical, and an overly compliant tendon can reduce the ability for the tendon to transmit force in the tendon-muscle complex. Any replacement to the tendon, either whole (prosthetic) or partial, must also replace this element in series with appropriate stiffness that will not degrade with large cycles. The stiffness of shape memory alloys (such as Nitinol) can be engineered through cold-working, hot-working, or aging to approximate the in vivo properties of the soft-tissue, such as by narrowing the hysteresis curve through cold-working (FIG. 5) to better approximate the energy storage capabilities of the tendon.

Furthermore, the surface finish of the Nitinol core 22 is critical to its biocompatibility and fatigue life. Prior to use, the Nitinol core 22 may be passivated to remove embedded surface contaminants that may have resulted from the manufacturing process to improve biocompatibility. The Nitinol can be coated with polytetrafluoroethylene (PTFE) for improved lubricity.

### Alternative Embodiments

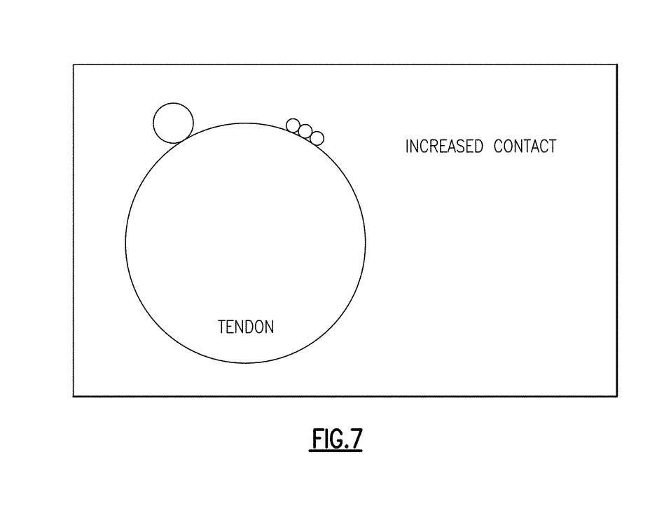

Other preferred forms of the invention include alterations to the format of the shape memory alloy core 22 of the suture 20. The poly-blended cover 24 will remain the same for each embodiment. The shape memory alloy core 22 can be provided in a ribbon format for increased surface area when pull-out risk is highest and fewer anchor points are desired. As shown in FIG. 6a, the shape memory alloy core 22 can be a single strand 26 (or filament) of circular wire for highly predictable and engineered material properties to match the desired application. As shown in FIG. 6b, the shape memory alloy core 22 can comprise a plurality of strands 26 (multi-filament) wound or braided together to provide strength properties similar to a larger single strand, with the advantage of greater contact area (FIG. 7).

### Individually Protected Strands

As shown in FIG. 6c, the final construct can also be provided as a plurality of single shape memory alloy strands 26 individually covered in a cover 24 and each individually wound or braided together to protect shape memory alloy strands 26 from issues associated with fretting when a large number of high strain cycles are expected. The individually protected strands 26 may also be coated in a protective coating of polytetrafluoroethylene.

### Martensitic and Superelastic Core Options

The suture 20 can have a shape memory alloy core 22 with a transition temperature (Af) higher than body temperature so that the suture 20 remains in the more pliable and easily worked martensitic phase during and after implantation. The suture 20 can also have an Af temperature below body temperature such that the suture 20 remains in the austenitic phase to utilize the superelastic properties of the shape memory alloy, such as when a knotless construct is needed.

### Shape Memory Alloy Engineered Cover

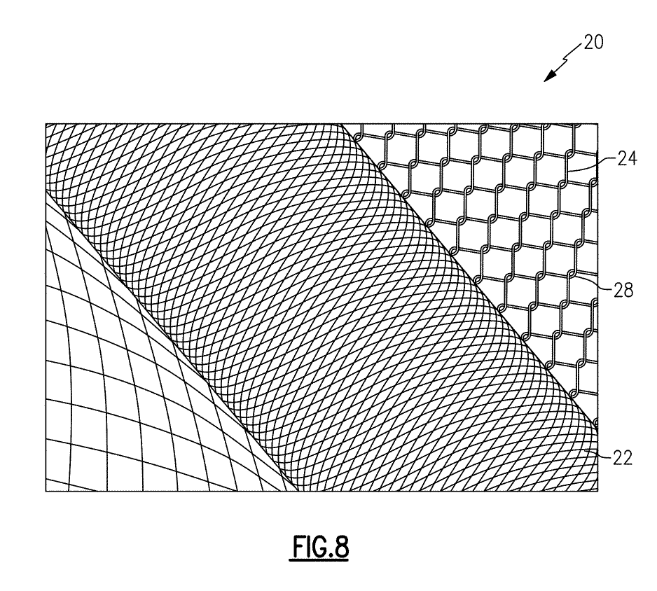

When an Achilles tendon is under load, it is subject to a “wringing” action. Because the gastrocnemius crosses the knee joint and a flexed knee can rotate, the part of the Achilles tendon that is derived from the tendon of the gastrocnemius can be variably twisted relative to the tendon of soleus (i.e., one tendon can exert a sawing action on the other). This complex rotatory action is further compounded by the shape of the talus. As shown in FIG. 8, when rotary action will be experienced by the soft-tissue (or bone) and suture construct, the resulting rotary action can be countered (or preferentially biased towards) by using a cover 24 including Nitinol threads 28 braided or woven into the cover 24 along with the polyester and/or polyethylene to resist the resultant torsion. The suture 20 can also contribute to the desired matching of the tensile soft-tissue properties engineered into the core 22, as described below. If there is a Nitinol core 22, it can be shape set to have a torsional bias. The Nitinol filaments 28 within the cover 24 may also be coated in polytetrafluoroethylene.

### Matching Soft-Tissue Properties

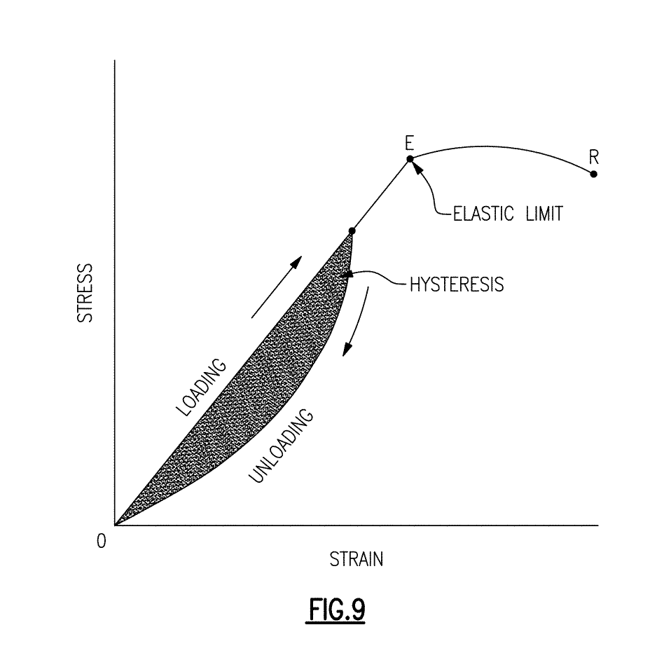

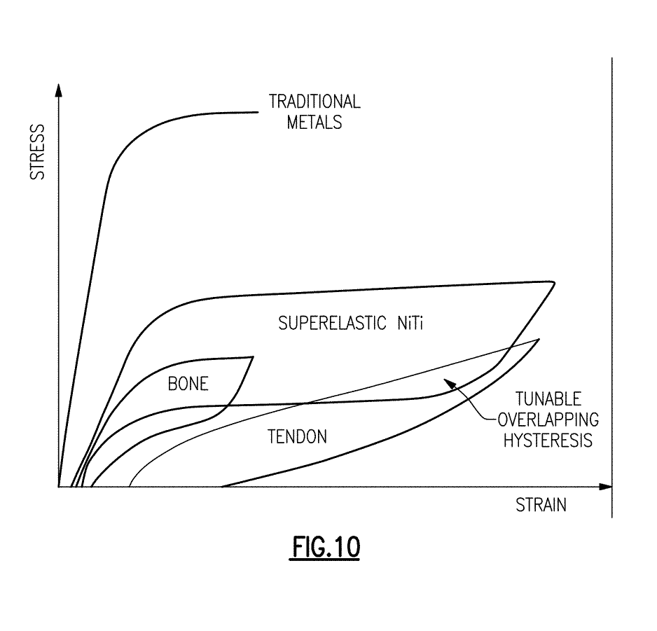

As shown in FIG. 9, tendons and ligaments exhibit a hysteresis between the loading and unloading phases representing a loss of stored energy. This hysteresis curve is of a similar shape to that of Nitinol. Most structural materials, like stainless steel or titanium alloys, do not have this unique hysteresis. To better match the properties of the target soft-tissue, the Nitinol core 22 of the suture 20 can be engineered through dimensional alterations, cold working (as described above), heat-treatment (aging and/or stress relieving and annealing, as described above) or the addition of filaments to the core 20 or the cover 24 to overlap the hysteresis curve of the target tendon or ligament (as shown in FIG. 10).

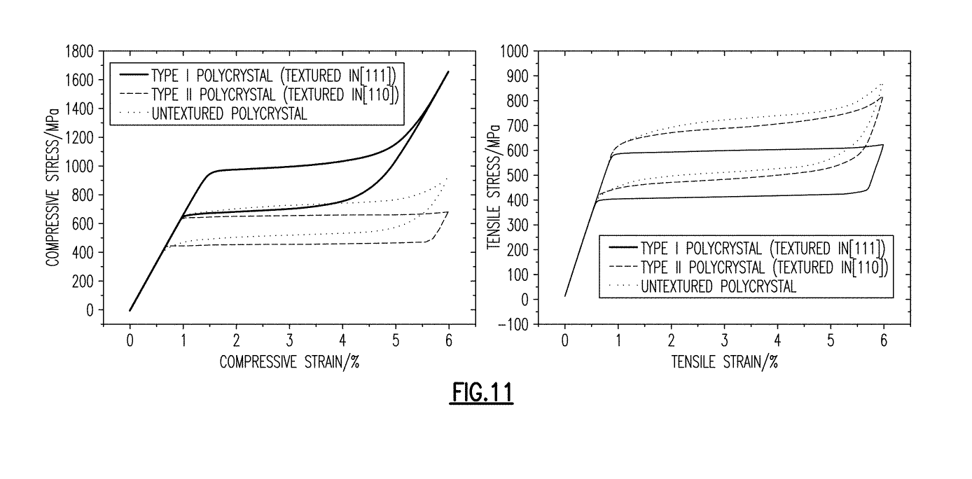

The strength and strain recovery ability of NiTi is highly dependent on the thermomechanical processing pedigree of the material from melted ingot thru the cogging, forging, rolling, drawing and intermediate heat treatment steps. The thermomechanical processing steps manifest themselves in various crystallographic texture orientations in the Nitinol. Because Nitinol is anisotropic, these crystallographic texture orientations have a profound effect on the material's shape recovery strain as it relates to loading direction and their tension/compression asymmetry. For example, when tested in either compression or tension, there are significantly different resistances to compression or tension, respectively, when the specimen is loaded either parallel ([111] crystallographic preferred direction) or perpendicular ([110] crystallographic preferred direction) to the rolling direction or to the texture of the principal grains resulting from hot and/or cold work of the base material (FIG. 11).

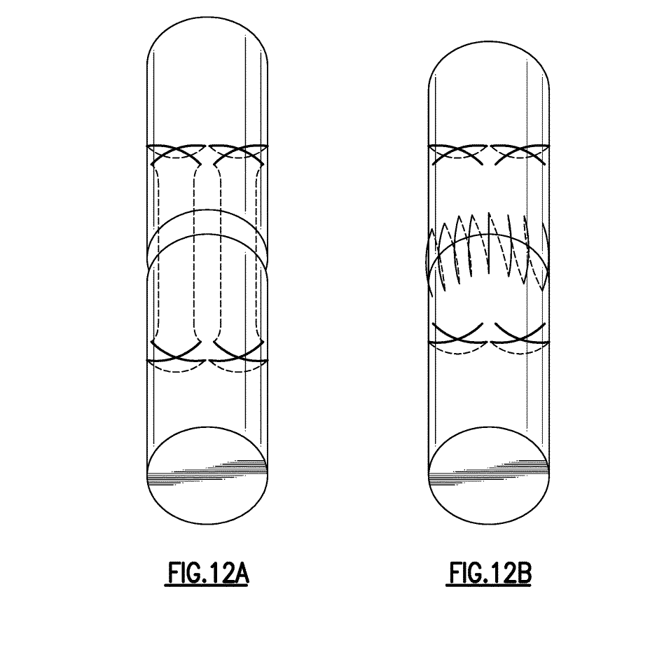

The present invention utilizes the preferred crystallographic texture in the Nitinol to be loaded with the fibers of the tendon-ligament repair construct to optimize its strength and shape recovery strain such that the underlying material is designed to best withstand the forces to which they are subjected. This can vary from region to region depending on how the individual regions of the construct are processed (see U.S. patent application Ser. No. 14/699,837). As shown in FIGS. 12a and 12b, a conventional repair for attaching loose ends of a tendon is to use grasps or loops to secure the suture in the soft-tissue and strands which cross the damage gap. A construct of the present invention can have different properties in the regions used as strands and the region used as loops in recognition of the different forces experienced in the two regions. In other words, the Nitinol maybe have more strength when looped circumferentially based on a circumferential crystallographic texture from the drawing process. Or, the Nitinol suture can be used parallel to the tendons' fibers to maximize the materials shape recovery and strength based on its thermomechanical texture which runs parallel to the tendon fibers.

### Tendon or Ligament Prosthesis

The current invention can also apply to the development of a biocompatible tendon or ligament prosthesis. The device can be a complete replacement (i.e. ACL or PCL reconstruction) or a partial replacement (i.e. when tendon lengthening is required, and an allograft may otherwise be utilized). The parallel nature of the cover 24 and the core 22, both of which contribute to the structural and mechanical properties of the device, make the invention an ideal candidate for seamless integration with existing or preserved soft-tissue.

### Tendon or Ligament Prosthesis—Spacer Fabric/Biological Mesh

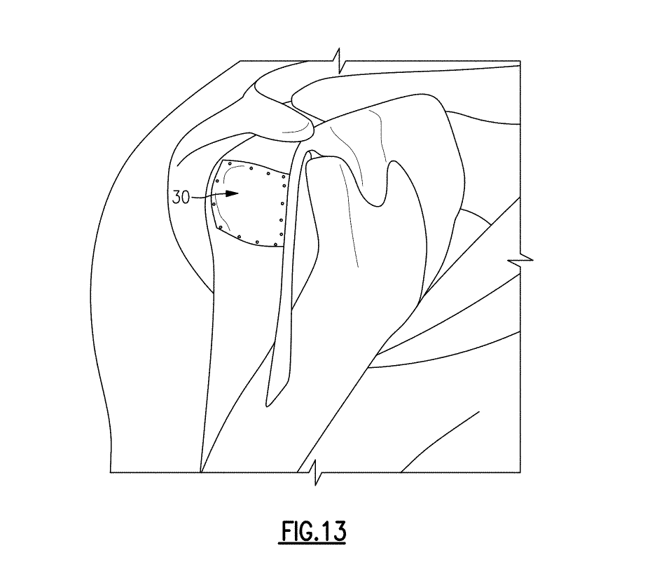

The tendon or ligament prosthesis can be highly engineered to match the target tissue properties. Meshes are used to surgically repair and/or reinforce ligaments, tendons and/or muscles. By way of example, but not limitation, meshes are commonly used to repair and/or reinforce the rotator cuff of the shoulder, the Achilles tendon in the heel, and other ligaments, tendons, and/or muscles throughout the body (e.g., the joint capsule of the hip, the joint capsule of the knee, etc.). These tissues may be injured or weakened due to trauma and/or degradation. Primary repair of the tissue may be effected with sutures to re-approximate the anatomy. However, these re-approximated tissues often fail to heal biologically, and the sutures may pull through the re-approximated tissue. In these situations, a mesh may be used to reinforce the repair. The mesh may be laid over or wrapped around the tissue and then sutured into place. The mesh takes up some of the load, thereby limiting stress on the damaged tissue during healing. FIG. 13 shows a mesh 30 used in the repair of a torn rotator cuff in a shoulder.

A biologic mesh or spacer fabric composed of a combination of shape memory alloy and polymeric fibers or biologic fibers may also be used. The biologic fibers can be silk fibers, collagen fibers, elastin fibers, etc. The biologic fibers can be used alone or in combination with polymeric and non-polymeric fibers and be made of Nitinol and other shape memory alloys to enhance tissue integration (refer to U.S. patent application Ser. No. 14/030,695). The spacer fabric can be woven in a tubular or cylindrical fashion to approximate the shape of a target replacement tissue (ligament or tendon) and utilized as the prosthetic tissue or as an augment to the tissue. This construct can be engineered to match the properties of the target tissue through a combination of the weaving process and the pedigree of the underlying shape memory alloy.

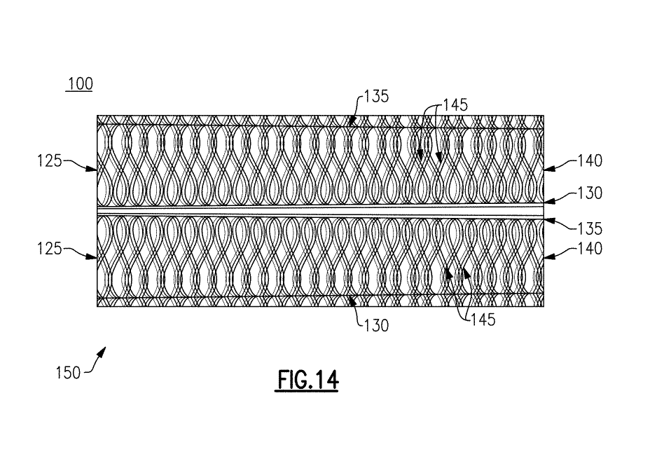

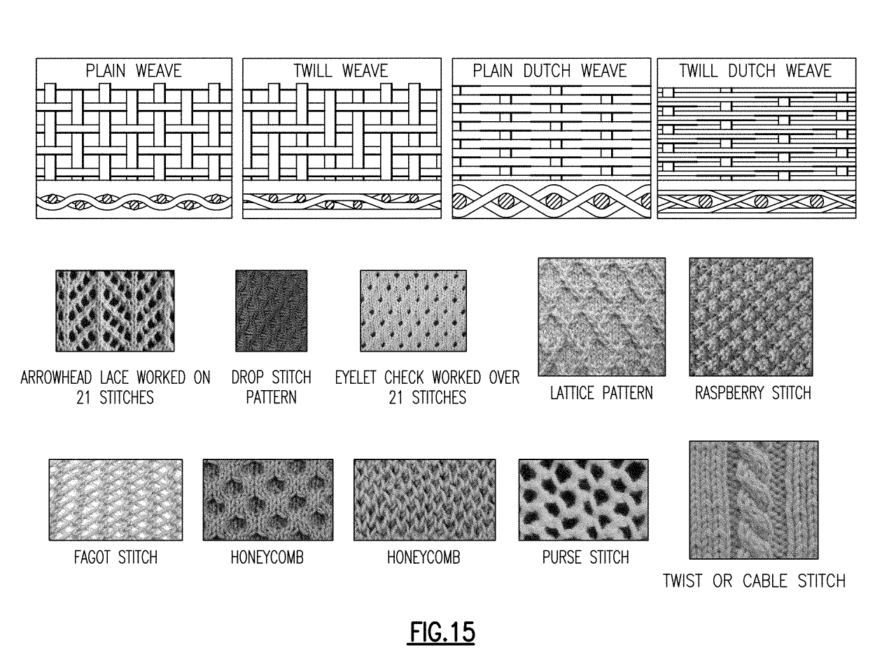

As shown in FIG. 14, by appropriately selecting (i) the composition and geometry of the fibers, (ii) the mesh pattern and pore size of a bottom layer 130 and a top layer 135, (iii) the orientation and spacing of interconnecting fibers 145 of an intermediate layer 140, and (iv) a thickness of the bottom layer 130, the top layer 135 and the intermediate layer 140, the material properties of a spacer fabric 125 (and hence a mesh 100) can be controlled to match the target tissue. By way of example, but not limitation, a thicker overall spacer fabric 125 manufactured using thinner filaments is generally more compliant than a thinner overall spacer fabric manufactured from thicker filaments (refer to U.S. patent application Ser. No. 14/030,695). Further, the shape memory alloy can be superelastic to simulate the mechanical properties of tendon, ligament, and muscles. As shown in FIG. 15, they can be knit, woven, and braided into various textile constructs including, not limiting to, plain weave, twill weave, plain dutch weave, twill dutch weave, and various other stitches and patterns.

### Axial and Circumferential Tendon and Ligament Fixation Inverse Stent

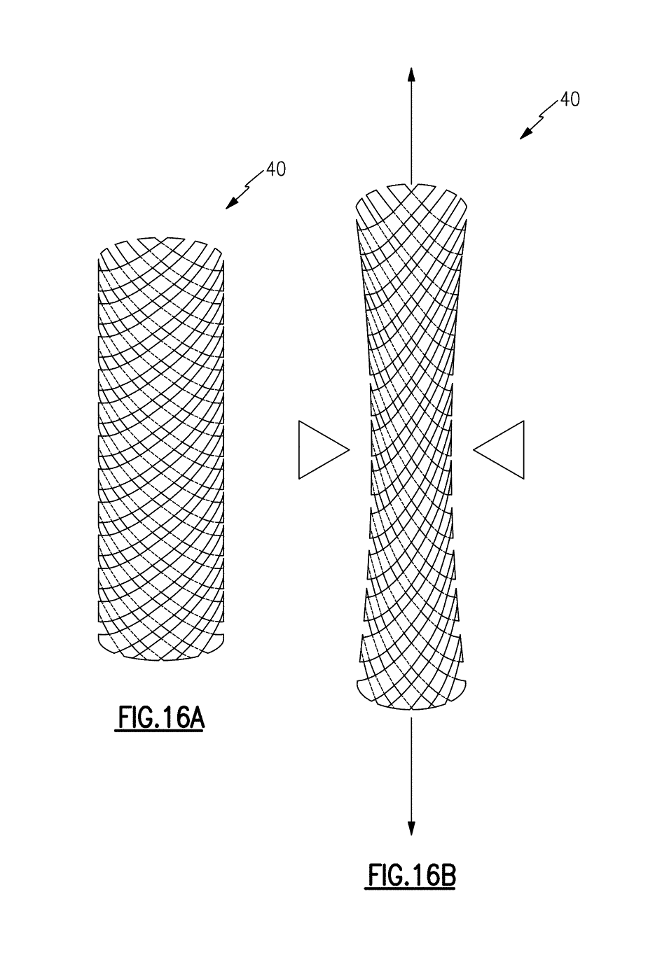

Tendons and ligaments which have been completely severed can also be secured with this technology. As shown in FIGS. 16a and 16b, a tendon/ligament (soft-tissue) repair device 40 can be engineered to compress radially when a proper delivery device is removed and then resist any tension in a manner similar to a “finger trap” such that gap formation is resisted and healing can proceed. This provides a consistent and repeatable mechanism to secure the soft-tissue that is not contingent on a knot technique and could allow the procedure to be done faster. The stiffness of the soft-tissue repair device 40 can be designed to be compatible to the target tissue by changing the dimensions of the repair device 40, i.e., the strut thickness, the strut width, the strut angle, and the strut frequency.

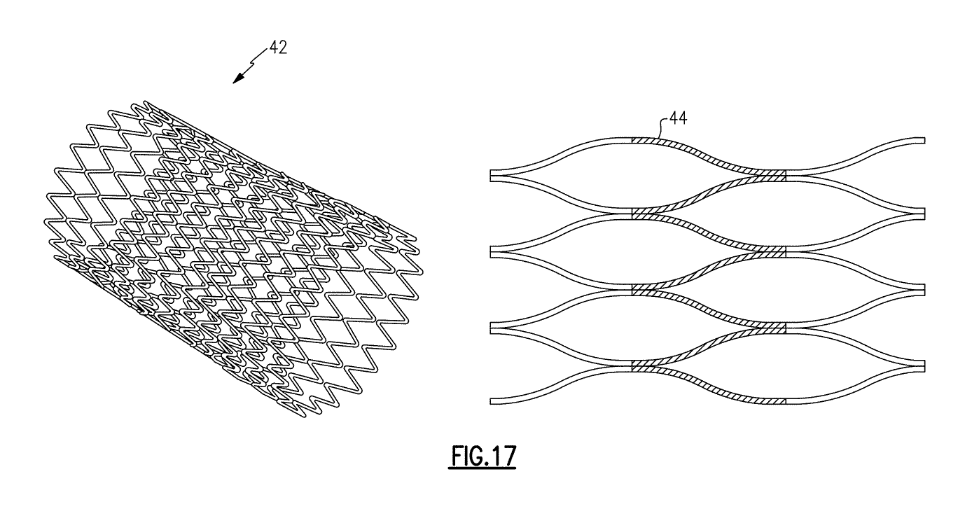

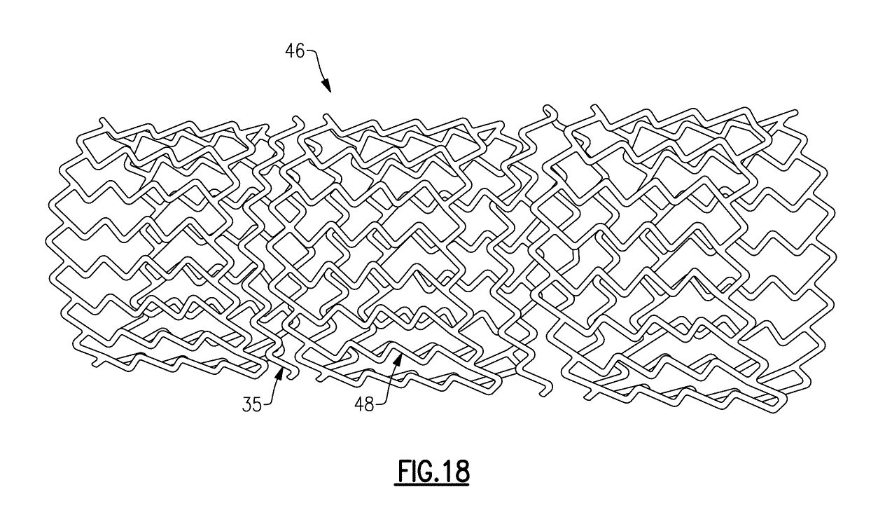

FIG. 17 shows struts 44 having a “Z” shape that make up the structure of the soft-tissue fixation device 42. FIG. 18 shows a soft-tissue repair device 46 having the conventional Z struts 35 that compress radially, and W struts 48 adjacent to and normal to the Z struts 35. The Z struts 35 radially compress, and the superelasticity of the W struts 48 resist pulling and the development of gaps in the repair. In addition to the traditional stent foreshortening effect which can be used to compress tendons together, the design of the soft-tissue repair device can be further enhanced to increase both radial compression and longitudinal resistance. If desired, the soft-tissue repair designs can have angular zigzags to help with torsional stability. That is, the soft-tissue fixation device 42 could have Z struts 35, W struts 48, and/or off center line zigzags to improve torsional stability.

The “finger trap” design also applies to a woven hollow cylinder that is delivered either stretched (FIG. 16B) or unstretched (FIG. 16A). The stretched format would inherently retract, maintaining the contact between ends of the tissue. This force would be improved by the inherent superelasticity of the shape memory alloy. The unstretched format would rely on the retraction of the weave as well as the superelasticity of the shape memory alloy to resist pulling apart.

The “finger trap” design can be made of an engineered spacer fabric utilized to match target tissue properties. The “finger trap” design can also be utilized during tendon or ligament transfer. The device can also be used to tubularize a ligament/tendon so that it can be pulled through a bone tunnel (such as during, but not limited to, ACL reconstruction), eliminating the step of whip stitching and tubularizing. The “finger trap” design can also be utilized during repairs requiring the reanchoring of a tendon/ligament to bone by grasping the loose end of the soft tissue and incorporating the device into a suitable bone anchor.

When an intravascular stent is deployed in an artery or vein, it radially expands and simultaneously shortens, which can cause the intravascular stent to undesirably tear the wall of the artery or vein as the intravascular stent foreshortens. Thus, in vascular applications, foreshortening can be problematic and undesirable. In the present invention, the struts of a tendon/ligament construct can be made with barbs or tangs to aggressively grip the surrounded tissue to pull the tendon ends on either side of the injury together, effectively compressing the tendon together when foreshortening occurs.

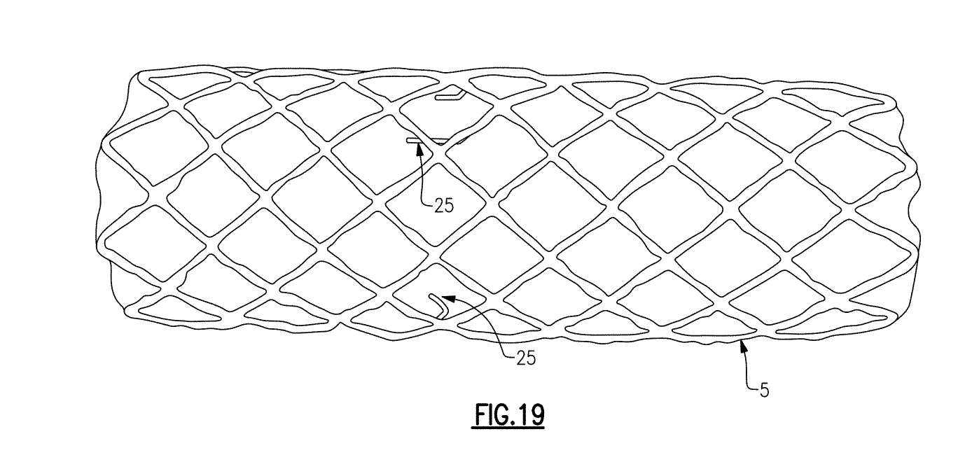

For example, FIG. 19 shows a Nitinol tendon/ligament fixation device 5 having integral barbs 25 for gripping the tissue of the host. The foreshortening effect is used to provide desired compression and pull-apart resistance. The tension applied by the shortening fixation device 5 to the tendon may better maintain close apposition of the ends of the tendon and better establish compression across the fusion zone during the healing process. Tendons have the ability to heal even when a gap exists. However, when this undesirable condition occurs, the end result is longer than the intended repair and could cause future issues. Therefore, maintaining contact throughout the healing process provides a more desirable result. The Nitinol fibers in either embodiment may be coated in polytetrafluoroethylene.

In one example, a suture filament is suitable for use as a suture or a ligature. The suture filament includes a cover formed of a plurality of braided fibers of polyester and/or ultrahigh molecular weight polyethylene. The cover includes a plurality of shape memory alloy filaments. A core of Nitinol is also included. The core is surrounded by the cover. The core is selected from the group consisting of a single filament of Nitinol, a multi-filament of Nitinol that is wound, braided or twisted, and a ribbon shaped filament of Nitinol. The core has a material condition of a martensitic shape memory alloy where an austenitic finish temperature is at or above body temperature such that the core remains in a martensitic phase after implantation or an austenitic shape memory alloy where the austenitic finish temperature is below body temperature such that the core maintains superelastic properties after implantation.

A suture filament is suitable for use as a suture or a ligature. The suture filament includes a cover formed of a plurality of braided fibers of polyester, and/or ultrahigh molecular weight polyethylene, and/or a plurality of shape memory alloy filaments. The suture filament includes a core of shape memory alloy that is surrounded by the cover.

In another embodiment according to any of the previous embodiments, the shape memory alloy is Nitinol.

In another embodiment according to any of the previous embodiments, the core is a single filament of the shape memory alloy.

In another embodiment according to any of the previous embodiments, the core is a multi-filament of the shape memory alloy. The multi-filament is wound, braided or twisted.

In another embodiment according to any of the previous embodiments, the core is a ribbon shaped filament of the shape memory alloy.

In another embodiment according to any of the previous embodiments, includes a plurality of suture filaments braided into a bundled, multi-filament super structure.

In another embodiment according to any of the previous embodiments, the core has a material condition of a martensitic shape memory alloy where an austenitic finish temperature is at or above body temperature such that the core remains in a martensitic phase after implantation or an austenitic shape memory alloy where the austenitic finish temperature is below body temperature such that the core maintains superelastic properties after implantation.

In another embodiment according to any of the previous embodiments, the suture filament includes a coating of polytetrafluoroethylene.

In another embodiment according to any of the previous embodiments, the cover includes the plurality of shape memory alloy filaments coated in polytetrafluoroethylene.

In another embodiment according to any of the previous embodiments, the plurality of braided fibers of the cover are polyester or ultrahigh molecular weight polyethylene.

In another example, a construct for tendon/ligament repair includes a body comprised of Z struts, W struts, and off center line zigzags and comprises a shape memory alloy.

In another embodiment according to any of the previous embodiments, the shape memory alloy is Nitinol.

In another embodiment according to any of the previous embodiments, the shape memory alloy is coated in polytetrafluoroethylene.

In another embodiment according to any of the previous embodiments, the construct includes internal barbs for grip.

In another embodiment according to any of the previous embodiments, the construct is woven or braided.

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A suture filament suitable for use as a suture or a ligature, the suture filament comprising:
a cover formed of a plurality of braided fibers of polyester and/or ultrahigh molecular weight polyethylene, and
a core of Nitinol, wherein the core is surrounded by the cover,
wherein the core is selected from the group consisting of:
a single filament of Nitinol,
a multi-filament of Nitinol, wherein the multi-filament is wound, braided or twisted, and
a ribbon shaped filament of Nitinol, and
wherein the core has a material condition of.

2. A suture filament suitable for use as a suture or a ligature, the suture filament comprising:
a cover formed of a plurality of braided fibers of polyester, and/or ultrahigh molecular weight polyethylene; and
a core of shape memory alloy, wherein the core is surrounded by the cover.

3. The suture filament as recited in claim 2, wherein the shape memory alloy is Nitinol.

4. The suture filament as recited in claim 2, wherein the core is a single filament of the shape memory alloy.

5. The suture filament as recited in claim 2, wherein the core is a multi-filament of the shape memory alloy, wherein the multi-filament is wound, braided or twisted.

6. The suture filament as recited in claim 2, wherein the core is a ribbon shaped filament of the shape memory alloy.

7. The suture filament as recited in claim 2, including a plurality of suture filaments braided into a bundled, multi-filament super structure.

8. The suture filament as recited in claim 2, including a coating of polytetrafluoroethylene.

9. The suture filament as recited in claim 2, wherein the cover includes the plurality of shape memory alloy filaments coated in polytetrafluoroethylene.

10. The suture filament as recited in claim 2, wherein the plurality of braided fibers of the cover are polyester or ultrahigh molecular weight polyethylene.

11. A construct for tendon/ligament repair comprising:
a body comprised of Z struts, W struts, and off center line zigzags; and
a shape memory alloy.

12. The construct for tendon/ligament repair as recited in claim 11, wherein the shape memory alloy is Nitinol.

13. The construct for tendon/ligament repair as recited in claim 11, wherein the shape memory alloy is coated in polytetrafluoroethylene.

14. The construct for tendon/ligament repair as recited in claim 11, including internal barbs for grip.

15. The construct for tendon/ligament repair as recited in claim 11, wherein the construct is woven or braided.
